(19) United States
(12) Patent Application Publication
(10) Pub. No.: US 2003/0113310 A1
(43) Pub. Date: Jun. 19, 2003

(54) Method for the treatment of obesity, overweight and fluctuations in blood insuline and/or glucose levels

(76) Inventors: Katrien Maria Jozefa Van Laere, Heteren (NL); Arie Gijsbert Nieuwenhuizen, Utrecht (NL)

Correspondence Address: YOUNG & THOMPSON, 745 SOUTH 23RD STREET 2ND FLOOR, ARLINGTON, VA, 22202

(21) Appl. No.: 10/015,582

(22) Filed: Dec. 17, 2001

(51) Int. Cl. A61K 38/51

(52) U.S. Cl. 424/94.5

(57) ABSTRACT

The present invention relates to a method of treating or preventing obesity, overweight, fluctuations in blood insulin levels and/or fluctuations in blood glucose levels in mammals. The method acording to the invention comprises the enteral administration to a mammal of an effective amount of a preparation containing an enzyme capable of converting an ingested carbohydrate or digestion product thereof into one or more absorbable components, wherein the total metabolic caloric value of the absorbable component(s) is less than the metabolic caloric value of the ingested carbohydrate or digestion product thereof. Thus the present invention effectively provides a method that allows complete digestion of ingested digestible carbohydrates whilst at the same time reducing the actual metabolic caloric value of said ingested carbohydrates.

Another aspect of the invention relates to a pill for oral administration provided with an enteric coating and containing 25 to 10.000 IU glucose isomerase per gram.

### 1. FIELD OF THE INVENTIONS

The present invention relates to method for the prevention or treatment of overweight, obesity or fluctuations in blood insuline and/or glucose levels in mammals, the method comprising the administration to a mammal of an enzyme capable of converting an ingested carbohydrate or a digestion product thereof into one or more absorbable components, wherein the total metabolic caloric value of the absorbable component(s) is less than the metabolic caloric value of the ingested carbohydrate or digestion product thereof. The invention also provides a preparation useful for such treatment.

### 2. BACKGROUND OF THE INVENTION

Methods for treatment or prevention of obesity, overweight and fluctuations in blood glucose and/or blood insulin levels known in the art often make use of foodstuffs with reduced caloric value; compositions stimulating metabolism, e.g. by inducing in vivo thermogenisis; or compositions providing in vivo inhibition of digestive enzyme activity. Many drawbacks are attached to the methods as described above. Especially low caloric diets are particularly undesirable due to the required change in consumption pattern and the adverse taste of many low caloric foodstuffs. The inhibition of digestive enzyme activity has the disadvantage that it often causes flatulence and that its efficacy is seriously influenced by dietary factors.

Like obese individuals, also subjects who desire to reduce blood glucose and/or blood insulin fluctuations have to carefully control their diet, e.g. by consuming limited amounts of carbohydrates or by consuming foodstuffs with a low carbohydrate content. The downsides are significant as, for example, low carbohydrate compositions often have a bad taste.

In view of the above considerations there is a need for a method which is capable of reducing the metabolic caloric value of ingested carbohydrate containing foodstuffs, but which does not rely on the inhibition of digestive enzyme activity. In addition there is also a need for a method which reduces the impact of ingested carbohydrate containing foodstuffs on blood insuline and/or blood glucose levels without using inhibition of digestive enzyme activity.

U.S. Pat. No. 4,396,602 describes a method of lowering the blood glucose level in mammals. The method comprises administering an enzyme capable of synthesizing sparingly-digestible saccharides from easily-digestible saccharides. The blood glucose level-lowering agent comprises the enzyme capable of synthesizing sparingly-digestible polysaccharides or oligosaccharides from easily-digestible saccharides, such as monosaccharides, oligosaccharides and polysaccharides. Enzymes providing the above effect are dextransucrase and cyclodextrin-synthesizing enzymes.

A major downside of the use of enzymes catalyzing the formation of indigestible polysaceharides and oligosaccharides is that ingestion of such enzymes may cause flatulence. The saccharides formed by the enzymes will not be absorbed by the intestinal cells and be transported to the colon, where these saccharides will be fermented. The fermentation of the oligo- and polysaccharides will result in excessive flatulence. Furthermore, it is questionable whether the conversion to indigestible polysaccharides is truly effective, in particular since a large fraction of the formed indigestible polysaccharides may be converted back to digestible polysaccharides. It is noted that a significant reduction of carbohydrate absorption will result in a rapid reappearance of appetite, which is likely to result in the early consumption of additional foodstuff.

The known use of enzymes in food preparations has mainly focused on the modification of food ingredients with the purpose of improving bioavailability of active components or of improving digestibility.

The increase of bioavailability of compounds by the addition of enzymes has been described in U.S. Pat. No. 6,099,844, which relates to a method of increasing the yield of extractable substance from a botanical in the gastrointestinal system of a human being comprising the step of ingesting an enzyme composition comprising a cellulase enzyme and a pectinase enzyme at approximately the same time as a botanical is ingested so that the cellulase and pectinase enzymes degrade the cellulosic and pectin constituents, respectively, contained in the ingested botanical, to obtain an enhanced quantity of extractable substance from the botanical.

U.S. Pat. No. 4,959,212 provides a non-toxic, oxidizing-energizing composition suitable for use as an accelerator of the carbohydrate oxidative degradation metabolic process or the direct oxidation of glucose. Such a composition is said to be effective to reduce the blood glucose concentration in a human body afflicted with diabetes. The composition optionally comprises an enzyme selected from the group consisting of fructose diphosphate aldolase, phosphofructokinase, hexokinase, glucokinase, glucose 6-phosphate dehydrogenase, glucose phosphate isomerase, D-glucose phosphotransferase and mixtures.

### 3. SUMMARY OF THE INVENTION

The present invention discloses a novel method for the prevention and/or treatment of obesity, overweight and fluctuations in blood glucose levels and/or blood insulin levels without the above mentioned drawbacks.

The current invention provides a method for the prophylactic and curative treatment of overweight, obesity and fluctuations in blood glucose levels and/or blood insulin levels comprising the enteral administration of a preparation containing an effective amount of an enzyme capable of converting an ingested carbohydrate or digestion products thereof into one or more absorbable components, wherein the total metabolic caloric value of the absorbable component(s) is less than the metabolic caloric value of the ingested carbohydrate or digestion product thereof. Thus the present invention effectively provides a method that allows complete digestion of ingested digestible carbohydrates whilst at the same time reducing the actual metabolic caloric value of said ingested carbohydrates.

In the present method digestible carbohydrates are fully digested. Hence, the method according to the present invention will not cause flatulence. The present method does not require a change in diet and will induce a satiety effect.

### 4. DETAILED DESCRIPTION OF THE INVENTION

The present invention provides a method of treating or preventing obesity, overweight, fluctuations in blood insulin levels and/or fluctuations in blood glucose levels, said method comprising the enteral administration of an effective amount of a preparation containing an enzyme capable of converting an ingested carbohydrate or digestion product thereof into one or more absorbable components, wherein the total metabolic caloric value of the absorbable component(s) is less than the metabolic caloric value of the ingested carbohydrate or digestion product thereof.

Enzymes

The enzyme used in the method according to the present invention is capable of converting an ingested carbohydrate into one or more absorbable components, wherein the total metabolic caloric value of the absorbable component(s) is below the metabolic caloric value of the ingested carbohydrate. According to a preferred embodiment, the enzyme is selected form the group of isomerases.

A main cause of overweight is the ingestion of vast amounts of glucose monomers or polymers including glucose monomers. According to a preferred embodiment the enzyme is capable of converting glucose into an absorbable component having a decreased metabolic caloric value compared to glucose. Especially advantageous is the conversion of glucose into fructose, thereby additionally providing the benefits of fructose, such as its thermogenic activity and appetite reducing properties. Hence, according to a particularly preferred embodiment, the enzyme is capable of the isomerization of glucose into fructose, i.e. glucose isomerase.

Metabolic Caloric Value Reduction

The term metabolic caloric value as used in the present invention encompasses the caloric value derivable from a carbohydrate by a mammal by complete oxidation of the carbohydrate. The metabolic caloric value of a carbohydrate can be determined on a theoretical basis and by analysis. When the theoretical metabolic caloric value of a carbohydrate is determined, the ATP required for the oxidation of the carbohydrate should be subtracted from the amount of ATP, which the complete oxidation of the carbohydrate would yield in a mammal.

In the case of glucose, absorption of orally ingested glucose in the duodenum requires about 0.5 mol ATP/mol glucose. The blood glucose can subsequently be converted either to fructose 1,6, diphosphate or can enter the gluconeogenesis via uridyldiphosphoglucose (UDPG), requiring 2 mol ATP/mol glucose. Fructose 1,6 diphosphate can subsequently be completely oxidized to form carbon dioxide and water, releasing a total of 38 mol ATP/mol glucose. Net gain of ATP per mol of exogeneous glucose oxidized is therefore 35.5 mol ATP. The metabolic caloric value of glucose is therefor 35.5 mol ATP/mol glucose.

The metabolic caloric value of fructose is 34.5 mol ATP/mol fructose. Fructose is, similar to glucose, absorbed in the intestine by a process requiring about 0.5 mol ATP/mol fructose. Fructose is generally metabolized essentially in the liver, where the enzyme fructokinase catalyses the phosphorylation of fructose into fructose-1-phosphate, requiring 1 mol ATP per mol of oral fructose. Subsequently, the fructose-1-phosphate is converted to glyceraldehyde phosphate (GAP) and dihydroxyacetone phosphate (DHAP) by the enzyme aldolase B. DHAP can be further degraded to pyruvate and enter the tricarboxylic acid cycle, or can be reconverted into glucose in the process of gluconeogenesis.

The conversion of orally ingested fructose to glycogen, requiring 2 mol ATP per mol of oral fructose (the conversion of glyceraldehyde to glyceraldehyde phosphate (GAP) requires hydrolysis of 1 mol ATP per mol of oral fructose and uridine triphosphate regeneration requires 1 mol ATP). Thus storage of orally ingested fructose as glycogen will require 3.5 mol ATP/mol fructose compared to 2.5 mol ATP/mol glucose.

The energy cost of storing fructose is thus about 9.2% of the caloric value of fructose, while the energy cost of storing glucose is about 6.5% of the caloric value of glucose. Consequently, fructose has a substantially lower metabolic caloric value than glucose.

The effect of glucose isomerase on the energy expenditure can be more accurately determined by measurement of energy expenditure by indirect calorimetry according to Scharz et al (Am J Physiol 1992;262(4 Pt 1):E394-401).

Glucose Isomerase

Several glucose isomerases with different characteristics are known in the art. According to a preferred embodiment a glucose isomerase is used which shows significant activity at the pH which normally occurs in the duodenum. Preferably the glucose isomerase has a pH optimum for converting glucose to fructose below 8.5, more preferably below 8, even more preferably below 7.5. The optimum is preferably at a pH above 4, even more preferably above 5.

Dosages

In accordance with the present invention the glucose isomerizing enzyme is suitably administered in an amount of between 10 and 100.000 international units (IU) per gram of the dosage. Preferably 0.5 to 1500 international units (IU) enzyme per kg body weight of the mammal are administered to the mammal per dosage. More preferably the dosage includes 1 to 750 IU enzyme per kg body weight, even more preferably 2 to 500 IU enzyme per kg body weight, most preferably 10 to 100 IU enzyme per kg body weight. Preferably the enzyme is glucose isomerase.

The glucose isomerase is preferably administered in a concentrated dosage form. The glucose isomerase can suitably be administered in a preparation preferably comprising between 25 and 10.000 IU glucose isomerase per gram, more preferable between 100 and 5000 IU glucose isomerase per gram, most preferably between 250 and 2500 IU glucose isomerase per gram.

Whenever the tern international unit (IU) is used in the present document this refers to the quantity of enzyme, which transfers 1 micromol glucose per minute to a carbohydrate having a lower caloric value than glucose, at pH 7.5, and 37° C. For example, 1 IU glucose isomerase refers to the quantity of glucose isomerase, which transfers 1 micromol glucose per minute to fructose at pH 7.5, and 37° C.

With glucose as a substrate, glucose isomerase activity can be assayed by the measurement of D-fructose produced during the isomerization reaction using the cysteine-carbazole method (CCM) which is based on the reaction of ketosugars with carbazole in acids to yield a purple product (Dische and Borenfreund, J. Biol. Chem. 192 (1951) 583).

Whenever the term dose or dosage is used within this disclosure, any dosage form is encompassed which can be administered enterally (e.g. orally), within a fairly narrow time span. Whenever reference is made to a certain quantity that is administered per dose or dosage, said quantity is preferably administered within one hour, more preferably within 15 minutes, even more preferably within 5 minutes.

Preparation

The term preparation within the spirit of the present invention refers to nutrional as well as pharmaceutical compositions. Pharamaceutical compositions may suitably include a pharmaceutically acceptable carrier. Pharmaceutical acceptable carriers are well known and described in the art. The preparation used in the present method can be applied in any suitable form, such as meals, bars, pills, capsules, gels, biscuits, drinks etc. According to a preferred embodiment the preparation is administered in a solid or semisolid dosage form, more preferably in the form of a pill, which term includes capsules, tablets, microparticles and microspheres.

The aforemention single solid or semisolid dosage form preferably has a weight between 0.1 and 30 grams, more preferably between 0.2 and 10 gram. When a pill is used to provide the enzyme, the pill preferably has a weight between 0.2 and 4 grams, even more preferably between 0.5 and 3 grams. A dosage can include one or more pills, however, preferably the dosage consists of 1 to 3 pills.

Many enzymes will not survive the acidic environment of the stomach. The enzyme used in the present method is preferably administered in an pill that is coated with a substance that can withstand the enteric environment (an enteric coating) or in another form that prevents the decrease of enzyme activity, e.g. by co-administering a buffer and/or by co-administering inhibitors of intestinal proteolytic enzymes. Alternatively or additionally, enzymes may be used which have reduced sensitivity to proteolytic breakdown or which are not or only partially affected by an acidic environment.

According to a particularly preferred embodiment the enzyme is administered in a solid or semi-solid dosage form with a coating that prevents the reduction of activity of the enzymes by stomach acid and/or stomach proteases. A delayed, post-gastric, release of the active enzymes in the small intestine (duodenum, ileum, jejunum) can be achieved by encasing the enzymes. One class of acid-resistant agents suitable for this purpose is that disclosed in Eury et al., U.S. Pat. No. 5,316,774. Effective enteric materials include polyacids having a pKₐ of from about 3 to 5. Examples of such materials are fatty acid mixtures, methacrylic acid polymers and copolymers, ethyl cellulose, and cellulose acetate phthalates. Specific examples are methacrylic acid copolymers sold under the name EUDRAGIT.RTM., available from Rohm Tech, Inc., Maiden, Mass., USA; and the cellulose acetate phthalate latex AQUATERIC.RTM., available from FMC Corporation, New York, N.Y., USA, and similar products available from Eastman-Kodak Co., Rochester, N.Y., USA.

Thermogenic Effect of Fructose

According to a preferred embodiment of the present method fructose is generated from ingested glucose. Fructose has been shown to provide an increased thermogenic effect compared to glucose. It is the inventors belief that an additional energy expenditure is required during the metabolisms of fructose, even further decreasing the metabolic caloric value of fructose. Ingestion of an enzyme capable of converting glucose into fructose, e.g. glucose isomerase, will therefore induce a thermogenic effect. This thermogenic effect contributes to the prevention or treatment of obesity or overweight. (Schwarz et al; Thermogenesis in obese women: effect of fructose vs. glucose added to a meal. Am J Physiol 1992;262(4 Pt 1):E394-401.)

Appetite Reducing Effect of Fructose

Furthermore, fructose ingestion is suggested to decrease food intake. Several mechanisms have been suggested to cause this appetite suppressing effect, however, the mechanism has not been elucidated. The suggested appetite reducing effect induced by fructose might be caused by the effect fructose has on gastric emptying. Fructose empties in a rapid, exponential fashion, while glucose empties in a more slowly, linear fashion. However, a more likely explanation for the appetite reducing effect of fructose can be found in the reduced fluctuation in plasma insulin levels and/or plasma glucose levels. Fructose ingestion leads to lower values of insulin in comparison to glucose ingestion. High insulin concentrations have been related to hunger feelings. Furthermore, there is evidence that glucose and fructose have a different impact on hepatic metabolism, which metabolism is believed to influence food intake. In animal studies it has been shown that jugular infusion of fructose, as opposed to glucose, decreases food intake when given before eating.

Ingestion of an enzyme converting glucose into fructose, e.g. glucose isomerase, will therefore reduce appetite and prevent hunger. The reduction of appetite is a highly desired impact for a preparation that is used in a method for the prevention and/or treatment of obesity or overweight. An enzyme capable of converting glucose into fructose is therefore especially useful in the method for the prevention and/or treatment of overweight and obesity.

Under normal physiological circumstances, ingested digestible di-, tri-, or polysaccharides are converted into monosaccharides in the acidic environments and/or by the carbohydrase activity in the mammalian intestinal tract. The monosaccharides are subsequently absorbed by the cells in the duodenum. Whenever reference is made in this document to ingested carbohydrate or a digestion product thereof, monosaccharides as well as digestible di-, tri-, oligo- or polysaccharides which can be converted into monosaccharides in the gastro-intestinal tract are meant.

Preferably, the absorbable carbohydrate formed by the enzyme used in the method according to the invention has a molecular weight between 75% and 125% of the molecular weight of the substrate, i.e. the ingested carbohydrate or digestion product thereof, preferably between 90% and 110%, even more preferably between 95% and 105%, especially between 99% and 101%. According to an especially preferred embodiment, the ingested carbohydrate or digestion product thereof is glucose or a di,- tri-, oligo- or polysaccharide containing glucose monose units and the absorbable carbohydrate is fructose.

In compositions meant for weight control, treatment or prevention of obesity or overweight often glucose has been (partially) replaced by fructose because of the above reasons. Although such diets provide at least part of the desired effects of fructose, still a vast amount of carbohydrates are consumed in such diets. Exclusion of “all” glucose comprising di-, tri-, and polysaccharides from foodstuff is impossible, in view of technical and commercial considerations. It is therefor desirable to accomplish the above advantageous effects of fructose, without the need of ingesting relatively large quantities of fructose. As explained herein before, this can be achieved by the ingestion of an enzyme capable of converting glucose to a monosaccharide of lower metabolic caloric value, e.g. fructose, such an enzyme preferably being a glucose isomerase.

Prevention and Treatment of Blood Glucose Fluctuations

The glycemic index is a measure for the effect of ingested foodstuff on blood glucose levels. The index gives a relative value for the blood sugar increase following the ingestion of the foodstuff. Fructose has a lower glycemic index (GI) value, compared to glucose (GI value glucose=100; GI value fructose=20). Additionally, fructose is first absorbed in the small intestine, then transported to the liver for conversion to glucose, its initial uptake is insulin independent.

Diabetics must manage their diet to maintain a normal blood glucose level: any increase in blood glucose will trigger an insulinemic response, creating an imbalance. This could lead to a serious insulin reaction or coma. Fructose, unlike glucose, does not cause a high initial glucose spike.

The use of enzymes capable of converting ingested glucose monosaccharides into fructose prevents abnormal insulin levels, reduces the insulinemic response of ingested glucose monosaccharide and provides a decreased fluctuation in blood glucose levels, all of which are highly desirable for subjects suffering form diabetes and associated diseases. Thus the present method may advantageously be used in the treatment or prevention of fluctuations in blood glucose levels and related disorders such as abnormal insulin levels, major fluctuations in blood insulin levels, insulinemic response after ingestion of foodstuff.

Cofactors/inhibitors

According to a preferred embodiment, the preparation further contains cofactors, e.g. minerals, that increase the activity of the enzyme. When glucose isomerase is used, preferably magnesium and/or cobalt is coadministrated. Magnesium can be included in the composition containing glucose isomerase in an amount between 10 mg and 5 g per dosage, more preferably between 30 mg and 1 g, even more preferably between 40 mg and 450 mg.

Combinations

In order to further improve the present method, the enzyme may be coadministrated with components capable of decreasing the absorption or digestion of ingested carbohydrates or digestion products thereof, e.g. carbohydrase inhibitors. Co-administration of such ingredients will increase the retention time of ingested and (partially) digested carbohydrate material in the duodenum, thereby increasing the amount of absorbable monosaccharide formed from the ingested carbohydrate per unit active enzyme. Preferred carbohydrate absorption inhibitors are gymnemic acid (e.g. obtainable from gymnema) or soluble indigestible fibers such as glucomannan and locust bean gum. Preferred carbohydrase inhibitors include plant derived polyphenols, selected form the group of catechins or derivatives thereof, anthocyanidins, proanthocyanidins, procyanidins and cyanidins, which are exemplary and preferably obtained green tea (Camellia sinensis) or grape (Vitis vinifera). The above components may be coadministered with the present enzyme in an amount of 0.001 to 1000 mg/IU of the enzyme, more preferably 0.01 to 100 mg/IU of the present enzyme.

Application

The enzyme is preferably administered to mammals having a body weight above 25 kg, more preferably to humans. Furthermore, the preparation can be advantageously used in the manufacture of a medicament for use in a method for the treatment and prevention of obesity or overweight, the method comprising the administration of an effective amount of glucose isomerase to a human.

A further objective of the present invention is to provide a cosmetic method for reducing or preventing the formation of body fat or keeping a lean body, comprising administering a therapeutically effective amount of a preparation comprising an enzyme capable of converting an ingested carbohydrate or digestion product thereof into one or more absorbable components, wherein the total metabolic caloric value of the absorbable component(s) is below the metabolic caloric value of the ingested carbohydrate or digestion product thereof.

The enzymes are preferably administered between 60 minutes before and 60 minutes after the ingestion of a significant amount of carbohydrates, e.g. at least 5 grams of carbohydrates. According to a further preferred embodiment, the enzyme is ingested prior to, during or shortly after a meal.

The enzymes are preferably ingested in the form of a pharmaceutical preparation or as a nutritional supplement.

### 5. EXAMPLES

### Example 1

### Pharmaceutical Composition

A tablet having an outside coating consisting of EUDRAGIT.RTM containing:

1 gram glucose isomerase (glucose isomerase 350 IGIU/gram, Sweetzyme T, Novozymes A/S, Denmark) and 150 mg magnesium chloride

### Example 2

### Nutritional Supplement

A nutritional supplement in the form of a gelatin capsule advertised to decrease the caloric value of ingested foodstuff and/or decrease blood glucose fluctuations comprising:

750 mg glucose isomerase (1500 IGIU/ml glucose isomerase (G-zyme, G993, obtained from Enzyme Bio-Systems, Beloit, USA)) and

250 mg Gymnema Sylvestre extract (comprising 15 wt % gymnemic acid)

### Example 3

### Fructose Formation Under Sub-Intestinal Conditions

To test the fructose forming properties of glucose isomerase under intestinal conditions mixtures of: 5 ml starch solution (5 ml 7.5 g Pacelli potato starch/100 ml 50 mM phosphate buffer; Paselli WA4 potato starch, AVEBE, Foxhol, The Netherlands), amylase (1 ml A6211, obtained from Sigma Chemie, Zwijndrecht) and brush border enzymes (0.2 ml, scraping of the inner wall of piglet small intestinal wall) were prepared. The mixtures were adjusted to pH 6.5 using 2 ml 50 mM phosphate buffers, which mimics the pH in the human intestine (pH 6-7.5). A mixture with and without 0.18 ml glucose isomerase (G-zyme, G993, obtained from Enzyme Bio-Systems, Beloit, USA) was incubated and the concentration of glucose and fructose was measured over time.

Table 1 gives the concentration glucose and fructose in the mixtures with and without glucose isomerase in time.

TABLE 1

| | Without | | | | |
|---|---|---|---|---|---|
| | glucose | With glucose isomerase | | | |
| | isomerase | | | Glucose + | Conversion rate |
| | Glucose | Glucose | Fructose | Fructose | glucose to |
| Time | concentration | concentration | concentration | concentration | fructose |
| (hours) | (g/l) | (g/l) | (g/l) | (g/l) | (%) |
| 0 | 2.6 | 1.8 | 0 | 1.8 | 0 |
| 0.5 | 6.3 | 4.1 | 1.5 | 5.6 | 26 |
| 1 | 10.6 | 6.8 | 3.0 | 9.8 | 31 |
| 3 | 13.8 | 9.2 | 4.6 | 13.8 | 33 |
| 4 | 15.5 | 10 | 5.6 | 15.5 | 36 |
| 6 | 15.6 | 12.3 | 7.0 | 19.4 | 36 |

From this study it can be concluded that fructose formation from glucose takes place under conditions as present in the small intestine.

### Example 4

### Fructose Formation Under Intestinal Conditions Using Pancreatine

To test the fructose forming properties of glucose isomerase under intestinal conditions, in the presents of pancreatine (including pancreas proteases), a mixture of 5 ml starch solution (7.5 g Pacelli potato starch/100 ml 50 mM phosphate buffer; Paselli WA4 potato starch, AVEBE, Foxhol, The Netherlands), 1.75 gram pancreatine (P1750, Sigma Chemie, Zwijndrecht), 475 mg cow bile and 0.15 ml brush border enzymes (scraping of the inner wall of piglet small intestinal wall) was prepared. The mixtures were adjusted to pH 6.5 using 2 ml 50 mM phosphate buffers, which mimics the pH in the human intestine (pH 6-7.5). A mixture with and without 0.2 ml glucose isomerase (G-zyme, G993, obtained from Enzyme Bio-Systems, Beloit USA) was incubated and the concentration of glucose and fructose was measured over time.

Table 2 gives the concentration glucose and fructose in the mixtures with and without glucose isomerase in time.

TABLE 2

| | Without | | | | |
|---|---|---|---|---|---|
| | glucose | With glucose isomerase | | | |
| | isomerase | | | Glucose + | Conversion rate |
| | Glucose | Glucose | Fructose | Fructose | glucose to |
| Time | concentration | concentration | concentration | concentration | fructose |
| (hours) | (g/l) | (g/l) | (g/l) | (g/l) | (%) |
| 0 | 1.5 | 1.5 | 0 | 1.5 | 0 |
| 1 | 3.7 | 3.1 | 0.7 | 3.7 | 19 |
| 3 | 5.9 | 4.5 | 1.2 | 5.7 | 21 |
| 7 | 9.3 | 7.1 | 3.4 | 10.5 | 32 |

From this study it can be concluded that fructose formation from glucose takes place in the presents of pancreatic proteases.

1. A method of treating or preventing obesity, overweight, fluctuations in blood insulin levels and/or fluctuations in blood glucose levels in mammals, said method comprising the enteral administration to a mammal of an effective amount of a preparation containing an enzyme capable of converting an ingested carbohydrate or digestion product thereof into one or more absorbable components, wherein the total metabolic caloric value of the absorbable component(s) is less than the metabolic caloric value of the ingested carbohydrate or digestion product thereof.

2. Method according to claim 1, wherein the enzyme is an isomerase.

3. Method according to claim 2, wherein the isomerase is glucose isomerase.

4. Method according to claim 2, wherein the method comprises administering 0.5 to 1500 international units (IU) enzyme per kg body weight of the mammal per dosage.

5. Method according to claim 1, wherein the absorbable component has substantially the same molecular weight as the ingested carbohydrate or the digestion product that the enzyme is capable of converting to the absorbable component.

6. Method according to claim 1, wherein the preparation is provided with a coating that prevents reduction of activity of the enzyme by stomach acid and/or stomach proteases.

7. Method according to claim 1, wherein said enzyme is administered to the mammal during a period of 60 minutes before and 60 minutes after ingestion of a carbohydrate and wherein the total amount of carbohydrate ingested during said period exceeds 5 grams.

8. Method according to claim 1, wherein the preparation further comprises one or more components selected from the group consisting of: carbohydrate absorption inhibitors, carbohydrase inhibitors and enzymes capable of synthesizing sparingly digestible saccharides from easily digestible saccharides.

9. A pill for oral administration provided with an enteric coating and containing 25 to 10.000 IU glucose isomerase per gram.
